(19) United States
(12) Patent Application Publication
(10) Pub. No.: US 2022/0168552 A1
(43) Pub. Date: Jun. 2, 2022

(54) Straberi EpiStamp Device

(71) Applicant: LESLIE NESBITT, New York, NY (US)

(72) Inventor: LESLIE NESBITT, New York, NY (US)

(21) Appl. No.: 17/108,095

(22) Filed: Dec. 1, 2020

(51) Int. Cl. A61M 37/00; A61B 18/14; A61B 17/3209; A61B 17/32

(52) U.S. Cl. CPC A61M 37/0015; A61M 37/0092; A61B 18/1477; A61B 2017/00199; A61B 17/320068; A61M 2037/0061; A61M 2037/0007; A61B 17/32093

(57) ABSTRACT

The present invention discloses an advance in enhanced stability, enhanced solubility, and enhanced uniqueness towards a micro needling stamper device by proposing an improved process by presenting a technique where skin care products are delivered by way of a needling device is presented. The device has a suction that operates in the casing of the needles. When the stamping of the microneedles occurs, this suction feature lift the skin from it's a regular lying surface. The suction feature promotes blood circulation which will promote cell repair and aide in regeneration. This vacuum like suction separates different layers of tissues, thus microtrauma from the needles is more confined to the layer desired; the epidermis. This also triggers an inflammatory response flooding the area with white blood cells, platelets, and other healing aids for the re-generation process.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

The present invention relates to skin care. More specifically, the present invention relates to the field of skin care product designed to prevent and treat atrophic acne scarring, loss of skin elasticity, post-inflammatory hyperpigmentation, stretch marks, cellulite, Acne vulgaris/Propionibacterium acnes, and other similar skin problems.

### Description of the Related Art

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Although compositions for skin care products operate through a variety of mechanisms, most provide only temporary moisture retention for improved hydration, transitory improvement in appearance and/or superficial relief of skin integrity.

Human skin is composed of three primary layers: the epidermis, dermis, and hypodermis. Each of these layers contains specific cell populations embedded within a complex physical and biochemical matrix, thereby providing the mechanical and functional organization necessary for the skin to maintain barrier function. It is well-known that the epidermis sloughs off millions of cells every day, creating an enormous demand for replacement cells. Central to this process, which operates continuously throughout an organism's lifetime, are the various population of progenitor stem cells and associated fibroblasts which provide biomechanical and trophic support. In order to meet this continual demand for new cells in the skin, progenitor stem cells proliferate and differentiate as part of the natural repair and regeneration mechanism of the body. Fibroblasts, which populate the dermis, provide vital trophic support, while producing important protein components of the skin.

Aging is a multifactorial phenomenon. The aging of the skin is mainly the result of one's genetic predisposition (known as chronological aging) and one's physiological reaction to environmental stresses (known as actinic aging). Chronological aging is largely genetically driven and appears to be largely linked to a reduction in antioxidant production. Actinic aging seems to be skin specific and is defined as the effect of the external environment on the skin's biological response. The skin response to actinic aging, which may be caused by sun and pollution exposure, as well as smoking, is typically associated with a lack of normal hydration, apparition of telangiectasia, sagging of the skin, and the appearance of fine lines and wrinkles.

Acne and rosacea are major diseases of the skin associated with sebaceous follicles on the skin. There are many treatments, but no cures for these diseases. Treatments for acne include antimicrobials, such as benzoyl peroxide, which kill or inhibit growth of Propionibacterium acnes bacteria shown to play a role in acne development; sebum modulating agents, such as retinoids, including tretinoin and isotretinoin, influence the rate of sebum production; keratolytic agents, such as salicylic acid, accelerates cell turnover and open hair follicles; anti-inflammatories, such as dimethylaminoethanol (DMAE), reduce redness and pain associated with acne lesions; cleansing agents, such as alcohols, open up the infundibulum and allow free sebum to exit the skin surface; anti-spot/pigmentation agents, such as ascorbic acid, prevent or treat pigmentation and color contrast on the skin; and anti-scar agents, such as copper peptides, help reduce the impact of scar formation from acne lesions. Rosacea can be treated with antibiotics, sulfur, sodium sulfacetamide, and retinoids.

It has also been proposed to treat acne by exposing the skin to electromagnetic radiation. The electromagnetic radiation typically includes wavelengths that are suitable to photochemically activate compounds, such as endogenous porphyrins, or their biochemical building blocks that are topically applied to the skin.

For example, McDaniel (US20030004499 and WO2003001894) teaches a method for dermatological treatment using narrowband, multichromatic electromagnetic radiation. A topical pre-treatment, such as an exogenous chromophore or a benefit agent may be used to enhance the penetration of light. The procedure may be repeated every 1 to 60 days.

Korman (US20020128695A1) teaches a method for high-energy photodynamic therapy of Acne vulgaris and seborrhea. The method includes illuminating an area of skin with high-intensity light, having a narrowband spectral characteristics of at least one of the following group of wavelengths consisting of 400 nm-450 nm (blue), 520 nm-550 nm (green) or 630 nm-670 nm (red) spectral range. The light source generates a high intensity, non-coherent light in the exact narrow spectral bands required for activation of the photodynamic reaction while filtering out harmful UV light. Pre-treatment with oxygen transporting compounds, such as perfluorocarbons, oxidative substances, such as hydrogen peroxide derivatives, keratolytic substances, and external photosensitizers, such as methylene blue, may be performed. The function of these pre-treatments is to release oxygen directly into the sebaceous glands and raise the efficiency of the destruction of Propionibacterium acnes.

Perricone (US20030009158A1) describes using topical treatments, such as glycolic acid, to enhance penetration of light or block light below the desired wavelengths (between 400 nm-590 nm). Such treatments may be applied prior to or during photo-treatment to increase light penetration into the skin. Chemical filters are used to remove light that is not within this desired range. Fat soluble fatty acid esters of ascorbic acid may be applied to the skin before, during, or after blue/violet light treatments.

Anderson (US20020099094) teaches light treatment of sebaceous gland disorders with 5-aminolevulinic acid (ALA) and photodynamic therapy. The ALA is described as metabolized via the porphyrin pathway. A metabolite infiltrates the skin to be treated. When intense light with a wavelength between 320 nm and 700 nm is delivered to ALA-treated skin, the metabolite (photoporphyrin IX) is excited and reacts with oxygen to produce singlet oxygen, thus, modulating sebaceous gland disorders such as acne.

Anderson (U.S. Pat. No. 6,183,773) describes a method of treating a sebaceous gland disorder by topically applying a chromophore or an “energy-activatable material” such as methylene blue, causing it to infiltrate into spaces of the skin, and exposing the skin to energy to photochemically activate the chromophore. The chromophore should have an absorption spectrum in the range of 600 nm to 1300 nm to minimize surrounding blood from absorbing light intended for the chromophore.

The preceding example illustrate conventional treatment of skin using electromagnetic radiation in a monotherapy approach. For example, in conventional treatment, the skin is exposed to electromagnetic radiation, perhaps only after a chromophore or a porphyrin precursor is topically applied thereto. The skin and the chromophore or a porphyrin precursor absorb radiation to raise the efficiency of the destruction of Propionibacterium acnes. As such, only a single biological pathway (thermal injury/recovery) is employed to affect a particular benefit. This is limited, since this solitary mechanism is prone to diminishing returns as the fluence, frequency or time of radiation, is increased. In many cases, saturation of the benefit is achieved beyond a certain fluence, frequency or time of treatment.

Accordingly, conventional practices are subject to several drawbacks. Firstly, electromagnetic radiation having a high energy density (fluence) is often utilized. The high energy density delivered may be unsafe for a lay user (e.g., a consumer) to use in a home setting. Furthermore, high fluence radiation tends to heat the skin to an uncomfortable temperature and therefore require that the skin be cooled during operation. For example, for devices that contact the skin, this uncomfortable heating may require that a skin-cooling system be built into the device itself, which can be expensive or limiting to the device design.

For other conventional practices, the fluence of radiation is too low to deliver adequate efficacy. Even if the patient goes through the inconvenience and expense of making frequent visits to a professional skin care specialist to receive multiple treatments, the results are often unsatisfactory. Furthermore, treatment with electromagnetic radiation alone does not impart protection from further aging-related degradation of the treated tissue that may result in the future.

The present invention discloses an advance methodology where the micro needling device is used for the treatment of atrophic acne scarring, loss of skin elasticity, post-inflammatory hyperpigmentation, stretch marks, cellulite, and Acne vulgaris/Propionibacterium acnes.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a novel and an improved method of skin micro needling device to treat multiple skin problems including, but not limited to, dark spots, wrinkles, stretch marks, scars, fine lines, acne, razor bumps, ingrown hair, keloid scars, spider veins, and loose skin.

It is further, the desirable object of the present invention is to provide a novel and improved method which uses sterile needles or microcrystalline needles, or needles constructed by natural mineral elements.

More particularly, it is a primary object of the invention to provide a new method for the stabilization and increased life of a skin treatment device which can be used without any internal or external side effects.

Another object of the invention is to provide a method of improving skin appearance in a subject, including a Digital Touch Screen Interface where a digital database function, is available to select needle depth.

It is moreover, the objective of the invention for preventing or reducing in a subject, signs of skin aging or of a skin condition or disorder, by applying an effective amount of composition treatments of the present invention to the skin of the subject, whereby illustrating the signs of skin aging or of a skin condition or disorder is prevented or reduced.

It is also the object of the invention to enhance the user's experience by providing an option to have manual or automatic mode operation of needles.

It is further the objective of the invention, to provide a new, more economical and stable assembly, which has the option to adjust speed and depth of needles for different treatment areas.

Still yet another object of the present invention is to provide a new and improved methodology for treatment of skin that address oxidative free radical-induced damage, which provides in the apparatus and methods of the prior art, some of the advantages thereof, while simultaneously overcoming some of the disadvantages normally associated therewith.

Still yet another object of the present invention is to provide a new and improved methodology for treatment of skin that address stretch markings, which provides in the apparatus and methods of the prior art, some of the advantages thereof, while simultaneously overcoming some of the disadvantages normally associated therewith.

This Summary is provided merely for purposes of summarizing some example embodiments, so as to provide a basic understanding of some aspects of the subject matter described herein. Accordingly, it will be appreciated that the above described features are merely examples and should not be construed to narrow the scope or spirit of the subject matter described herein in any way. Other features, aspects, and advantages of the subject matter described herein will become apparent from the following Detailed Description, Figures, and Claims sections.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention and explain various principles and advantages of those embodiments.

FIG. 1 illustrates the complete front and side view, as per preferred embodiments of the invention.

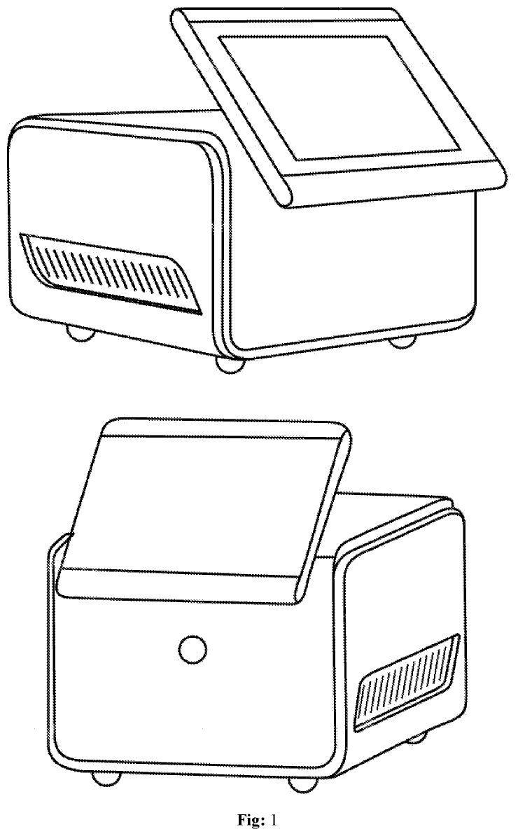

FIG. 2 illustrates stamper, as per preferred embodiments ofthe invention.

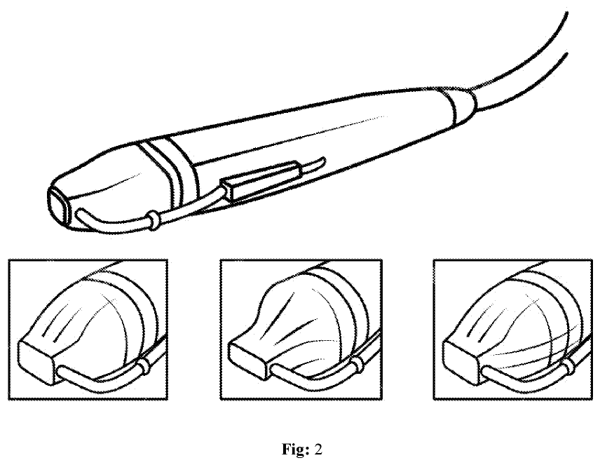

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Acne vulgaris, or commonly known as “acne”, is a skin condition that causes spots and often results in unsightly scar formation, which is not only disfiguring, but can be psychologically disturbing. Acne can vary from mild to moderate to severe and usually affects the skin of the face, back and chest. Acne affects more than four in five teenagers but it is much less common later in life. However, about one in 20 women and one in 100 men, ages between 25 to 40 years old, continue to be affected by acne, or develop it at this later age (“late-onset acne”). During puberty, both men and women produce more of the male hormone testosterone. This increases the production of sebum in skin, causing it to become greasy and encouraging spots to form. It is thought that this is one of the main reasons most teenagers develop acne during puberty. Acne starts to develop when hair follicles in the skin become blocked with the natural oil (sebum) produced by skin and dead skin cells. Each follicle is connected to a sebaceous gland that lies just underneath the surface of skin, normally sebum travels up the follicle and out through pores on the surface of the skin. However, if too much sebum is produced, and dead skin cells get trapped in the pores and a ‘spot’ will form, this can either be a whitehead or a blackhead. Sometimes the bacterium, Propionibacterium acnes, which normally lives on the surface of the skin, causes inflammation (but not infection) in the hair follicles. If the inflammation develops near the surface of the skin, red or yellow spots (pustules) can form. Deeper inflamed lesions (nodules and cysts) can form if the infection is closer to the hair root. In very severe acne, cysts may join together to form even larger, deeper inflamed lesions (Acne conglobata), but this is rare. Propionibacterium acnes and lesions can also be secondarily infected with Staphyloccocus aureus.

Assessing the grade of acne can help in the choice of the course of treatment that will be most effective. The most commonly prescribed treatments include: benzoyl peroxide; azelaic acid; retinoids, such as tretinoin and isotretinoin; antibiotic lotions, such as erythromycin or clindamycin, can help to control the Acne vulgaris/Propionibacterium acnes bacteria on the skin.

More recently, acne managed with blue light therapy has been proposed as an alternative form of treatment. The light may either be used alone or in conjunction with a topical modality. The acne blue light therapy has been shown to be effective in treating active lesions, as well as, other sebaceous glands that are not actively producing lesions. It has been reported that some of the violet light wavelength present in the visible spectrum of sunlight (in the range 415-430 nm) activates a porphyrin (Coproporphyrin III) in Propionibacterium acnes, which causes damage and ultimately, kills the bacteria by releasing singlet oxygen. Results have shown that the use of light therapy for three consecutive days has been shown to reduce the bacteria in the pores by 99.9%. Since there are few porphyrins naturally found in the skin, the treatment is believed to be safe except in patients with porphyria, although eye protection is used due to light-sensitive chemicals located in the retina. The light is usually created by superluminous LEDs. This form of treatment has been approved by the FDA for some lightwave systems. Overall improvement on average of 76% to 80% of patients occurs over three months; most studies show that it performs better than benzoyl peroxide and the treatment is far better tolerated. However, approximately 10% of users see no improvement.

Other disfiguring and psychologically disturbing skin conditions include psoriasis and contact dermatitis. Contact dermatitis presents as a localized rash or irritation of the skin and is caused by contact with a foreign substance. Inflammation of the affected tissue is present in the epidermis and outer dermis and the itchy rashes may take several days or weeks to fade and heal. Psoriasis is a more serious and chronic skin condition where skin cells reproduce about 10 times faster and exist in a far higher density than in normal skin, thus resulting in raised, scaly skin patches or plaques. The exact cause of psoriasis is not known, however, flares can be triggered by infection, stress, changes in temperature or climate, skin injury, some prescribed medicines, and alcohol intake. Psoriasis is typically treated by prescribed topical creams or gels (corticosteroids, vitamin D and vitamin A derivatives); light treatment or phototherapy, comprising of exposure to the affected skin area with either ultraviolet A (UVA) light alone or with psoralen (PUVA), in order to sensitize the skin to UV light; and/or oral medications, such as methotrexate and etretinate, which inhibit skin cell growth and inflammation.

Current products on the market either do not effectively address aging skin or have skin irritating side effects. The inventors of proposed invention, however, have discovered a unique combination of ingredients that work in a synergistic relationship with one another to effectively address the signs of early, moderate, and advanced skin aging.

The present invention is directed to an improved system and procedure for a stabilized cosmetic dermatological assembly which works with digital interface and focuses on addressing problems for the treatment of atrophic acne scarring, loss of skin elasticity, post-inflammatory hyperpigmentation, stretch marks, cellulite, and Acne vulgaris/Propionibacterium acnes.

The present invention discloses a new method for the stabilization and an enhanced formula wellness product with a micro needing device for the treatment of atrophic acne scarring, loss of skin elasticity, post-inflammatory hyperpigmentation, stretch marks, cellulite, Acne vulgaris and other similar skin issues.

The invention as per its preferred embodiments presents a new method where the skin care product is coupled with technology. The technique works where an option to use or not use ultrasound waves or radio frequency is presented. Furthermore, as per preferred embodiments an option to use sterile needles or microcrystalline needles or needles constructed by natural mineral elements, is also presented.

A Digital Touch Screen Interface as per its further embodiments is presented with a digital database finction to select needle depth. The system further presents an option to have manual or automatic mode operation of needles. The option to adjust speed and depth of needles for different treatment areas of the skin is also presented.

In certain embodiments, the present invention can decrease the amount of internal oxidation and/or external oxidative damage in a cell. In other aspects, the method can further increase collagen synthesis in a cell. The compositions can also reduce skin inflammation, such as by reducing inflammatory cytokine production in a cell. Non-limiting examples of such cells include human epidermal keratinocyte, human fibroblast dermal cell, human melanocytes, three dimensional human cell-derived in vitro tissue equivalents comprising human keratinocytes, human fibroblasts, or human melanocytes, or any combination thereof (e.g., combination of human keratinocytes and human fibroblasts or a combination of human keratinocytes and human melanocytes).

Also disclosed is a method of lightening skin or evening out skin tone pigmentation and texture, by comprising of applying the compositions of the present invention to the skin. The method can further comprise of identifying a person in need of lightening skin or evening out skin tone. The methods can further include inhibiting melanogenesis in a skin cell, inhibiting tyrosinase or tyrosinase synthesis in a skin cell, or inhibiting melanin transport to keratinocytes in a skin cell. The method can act as an alpha-melanin stimulatory hormone antagonist. The method can even out pigmentation of the skin. In a non-limiting aspect, skin lightening can include reducing the appearance of an age spot, a skin discoloration, or a freckle by topical application of the composition to the skin having an age spot, a skin discoloration, or a freckle, etc.

Also disclosed is a method of treating hyperpigmentation by comprising of applying the method of use of the present invention to the skin. The method can also comprise of identifying a person in need of treating hyperpigmentation. Additional methods, contemplated by the inventor, include methods for reducing the appearance of an age spot, a skin discoloration, or a freckle, reducing or preventing the appearance of fine lines or wrinkles in skin, or increasing the firmness and texture of skin.

In a particular embodiment, there is disclosed a method of reducing the appearance of a skin condition by comprising topically applying any one of the compositions described in this specification to the skin condition, wherein application of the composition to skin condition reduces the appearance of the skin condition. The skin condition can be any one of those described in this specification. In a particular aspect, the skin condition is a fine line or wrinkle, uneven skin tone, or an age spot. The skin condition can be located on facial skin, arm skin, leg skin, chest skin, abdominal skin, back skin, etc.

In yet another embodiment, there is disclosed a method of increasing the firnness of skin texture by comprising topically applying any one of the compositions described in this specification to skin in need thereof (a non-limiting example of which can be sagging skin, aged skin, skin that has reduced elasticity, skin that has skin cells having inadequate amounts of collage, fibronectin, or laminin or all of such proteins, etc.), wherein topical application of the composition to skin increases the firmness of skin texture. The composition can be used on facial skin, arm skin, leg skin, chest skin, abdominal skin, back skin, etc.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate the modifications that were made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressively recited in each claim. Rather, as the following claims reflect, inventive subject matter lies in less than all features of a single disclosed embodiment. Thus, the following claims are hereby incorporated into the Detailed Description, with each claim standing on its own merit as a separately claimed subject matter.

I: A stable Stamp Micro needling Stamper Device comprising:
a) Micro needling device with option to use or not use ultrasound waves, or radio frequency;
b) Micro needling device with option to use sterile needles or microcrystalline needles, or needles constructed by natural mineral elements;
c) Micro needling device with Digital Touch Screen Interface;
d) Micro needling device with digital database with function to select needle depth;
e) Micro needling device with option to adjust speed and depth of needles for different treatment skin areas

II: A skin treatment assembly for the treatment of including, but not limited to, atrophic acne scarring, loss of skin elasticity, post-inflammatory hyperpigmentation, stretch marks, cellulite, Acne vulgaris/Propionibacterium acnes wherein:
a) Suction feature is attached to the needles that adheres to the skin and lifts up the skin into the needle unit to reduce damage to underlying skin;
b) A button on device handle to control needle operation;
c) A non-slip grip and wide handpiece is presented;
d) Disposable needle cartridges;
e) Multiple needle size options;
f) Twist-lock motion needle application;
g) Wide and large handpiece for faster skin area coverage and;
h) Stamping mechanism is presented.

1. The method of reducing the appearance of a skin condition comprising by topically applying the procedure of claim I to the skin condition, wherein topical application of the composition to skin condition reduces the appearance of the skin condition, wherein the skin condition is a fine line, wrinkle, acne, uneven skin tone, or an age spot;

2. The method of claim I, wherein the skin condition is located on facial skin;

3. A method of increasing the firmness of skin texture comprising by topically applying the Micro needling Stamper Device to skin in need thereof, wherein topical application of the composition to skin increases the firmness and texture of skin;

4. The method of claim I, wherein the skin in need thereof comprises a fine line or wrinkle;

5. The method of claim I, wherein the composition is applied to facial skin and;

6. The method of increasing collagen, fibronectin, and/or laminin production in a skin cell comprising by topically applying the composition of claim I to a skin cell that is in need of collagen, fibronectin, and/or laminin production, wherein the topical application to the skin cell increases collagen, fibronectin, and/or laminin production in the skin cell.
